(19) United States
(12) Patent Application Publication
(10) Pub. No.: US 2023/0414640 A1
(43) Pub. Date: Dec. 28, 2023

(54) STABLE AQUEOUS COMPOSITION FOR PRETERM TO PROMOTE EARLY POSTNAL GROWTH

(71) Applicant: SOCIETE DES PRODUITS NESTLE S.A., Vevey (CH)

(72) Inventors: JELENA BUNCIC-MARKOVIC, St-Prex (CH); YIPU CHEN, La Tour-de-Peilz (CH)

(21) Appl. No.: 18/255,757

(22) PCT Filed: Dec. 2, 2021
(86) PCT No.: PCT/EP2021/083927

§ 371 Date: Jun. 2, 2023

(30) Foreign Application Priority Data
Dec. 4, 2020 (EP) 20211796.6

(51) Int. Cl. A61K 31/702; A23C 9/20; A61P 3/02

(52) U.S. Cl. CPC A61K 31/702; A23C 9/203; A61P 3/02

(57) ABSTRACT

The present invention concerns a stable aqueous composition comprising at least one human milk oligosaccharide for use in use in promoting postnatal growth in preterm infants as early as 3 weeks after supplementation, wherein the composition is given as soon as possible after birth.

### FIELD OF THE INVENTION

The present invention concerns a stable aqueous composition comprising at least one human milk oligosaccharide for use for use in use in promoting postnatal growth in preterm infants as early as 3 weeks after supplementation, wherein the composition is given as soon as possible after birth. The invention also relates to the use of such aqueous composition as a milk fortifier or supplement.

### BACKGROUND OF THE INVENTION

During development, especially in the first few years of life, children show interesting patterns of neural development with a high degree of neuroplasticity. The relation of brain growth and development and cognitive development is poorly understood and an area of growing research.

Some new development intends to prove a link between brain growth and cognitive development in infants and particularly in preterm infants or extremely low gestational age newborns (J. Pediatr. 2009; 155:344-9).

The importance of oligosaccharides which are contained in human breast milk (human milk oligosaccharides, HMOs) is well recognized in the scientific community as being key to support digestive health, gut and/or mucosal maturation, and/or immune maturation in infants. Accordingly, where the feeding of an infant is deprived of such nutrients (as the infant receives infant formulas not containing HMOs) or where the amount of HMOs in the human breast milk or formula the infant receives are not adequate to his needs, a composition providing HMOs in the form of a supplement to be administered to the infant or dissolved into his feeding would be desirable.

In particular, an aqueous liquid composition comprising HMOs would be needed.

WO 2014/043368 and WO 2013/057049 propose methods and compositions for improving brain growth and cognitive development and for enhancing memory functions in individuals by administering human milk oligosaccharides. However, these methods and compositions are not specifically suited for use infants or preterm infants in the very early postnatal period.

Thus, there is still a need for improved nutritional compositions for use in promoting brain growth and development in infants, and, in particular, in pre-term infants with low-birth weight (LBW) and infants who experienced intrauterine growth retardation (IUGR) or who suffered from growth stunting because of malnutrition, such as suboptimal intra-uterine nutrition, and/or disease.

Liquid aqueous compositions for enteral nutrition need to be microbiologically safe for preterm infants.

Accordingly, it is an object of the present invention relates to a method for feeding low birthweight infants (LBW infants), very low birthweight infants (VLBW infants), extremely low birthweight infants (ELBW infants) and preterm infants for use in use in promoting postnatal growth in preterm infants as early as 3 weeks after supplementation, wherein the composition is given as soon as possible after birth.

In the present study improved early postnatal growth has been clinically demonstrated for the first time especially in head growth, which is well-established determinant of longitudinal cognitive development of preterm infants.

### SUMMARY OF THE INVENTION

Accordingly, in one aspect the present invention provides an aqueous composition comprising at least one human milk oligosaccharide for use in use in promoting postnatal growth in preterm infants as early as 3 weeks after supplementation, wherein the composition is given as soon as possible after birth. The postnatal growth is increase in head circumference and/or length. The head circumference/growth is well-established determinant of longitudinal cognitive development of preterm infants. As human milk oligosaccharides are generally non-nutritive compounds not known to impact weight gain, results we have seen on these oligosaccharides supporting length and head growth in preterm infants as early as three weeks are novel findings.

In another aspect, the present invention provides an aqueous composition as a milk fortifier or supplement for preterm infants who were low birthweight infants (LBW infants), very low birthweight infants (VLBW infants), extremely low birthweight infants (ELBW infants).

In another aspect the present invention provides an aqueous composition of pH ranging between 4 and 7 comprising 2′-FL and LNnT as the only human milk oligosaccharides at ratio 10:1 for use in use in promoting postnatal growth in preterm infants as early as 3 weeks after supplementation, wherein the composition is given as soon as possible after birth. The postnatal growth is increase in head circumference and/or length.

### BRIEF DESCRIPTION OF THE DRAWINGS

Additional features and advantages of the present invention are described in, and will be apparent from, the description of the embodiments which are set out below with reference to the drawings in which:

FIG. 1: Anthropometric z-scores from FEF Day 1 until Discharge in HMO and Placebo groups.

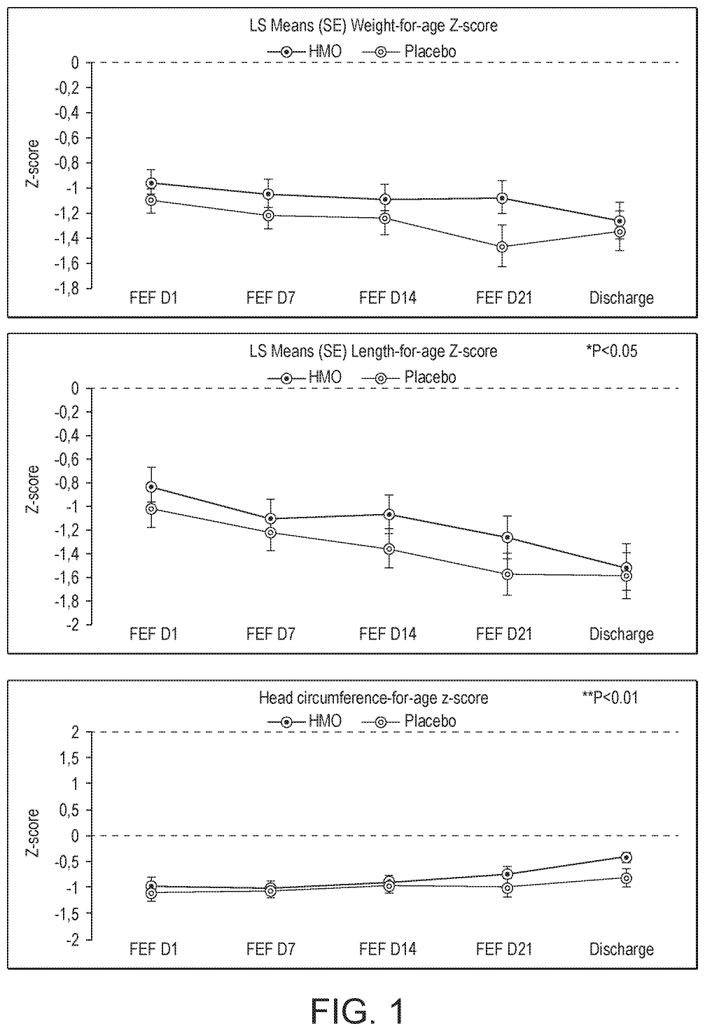

### DETAILED DESCRIPTION OF THE INVENTION

### Definitions

A “preterm” or “premature” means an infant or young child who was not born at term. Generally it refers to an infant or young child born prior 37 weeks of gestation.

An “infant having a low birth weight” means a preterm having a body weight below 2500 g (5.5 pounds) either because of preterm birth or restricted fetal growth. It therefore encompasses:
infant or young child who has/had a body weight from 1500 to 2500 g at birth (usually called “low birth weight” or LBW)
infant or young child who has/had a body weight from 1000 to 1500 g at birth (called “very low birth weight” or VLBW)
infant or young child who has/had a body weight under 1000 g at birth (called “extremely low birth weight” or ELBW).

Within the context of the present invention, the term “monosaccharide” indicates carbohydrates containing from 3 to 6 carbon atoms. They can be polyhydroxy aldehydes or polyhydroxyketones depending on whether they comprise either an aldehyde or a ketone group, along with —OH substituted carbons in a chain. Polyhydroxy aldehydes are called “aldoses”. Polyhydroxyketones are called “Ketoses”. Non limiting examples of 6 carbon monosaccharide (hexose) are: allose, altrose, glucose, mannose, gulose, idose, galactose, talose, psicose, fructose, sorbose and tagatose. Non limiting examples of 5 carbon monosaccharide (pentose) are: ribose, arabinose, xylose, lyxose, ribulose and xylulose.

Within the context of the present invention, the term “oligosaccharide” indicates a linear or branched saccharide polymer containing a small number (typically two to ten) of simple sugars (5 or 6 membered monosaccharides as above defined). Non-limiting examples of such oligosaccharides are: “fucosylated oligosaccharide” that are based on lactose, meaning an oligosaccharide having at least one fucose residue and a glucose at the reducing end. Some examples are 2′-FL (2′ fucosyllactose), difucosyllactose (DFL, also known as LDFT, Lactodifucosyltetraose), LNT (lacto-N-tetraose), para-lacto-N-neohexaose (para-LNnH), LNnT (lacto-N-neotetraose)and any combination thereof.

Within the context of the present invention, the expressions “fucosylated oligosaccharides comprising a 2′-fucosyl-epitope” and “2-fucosylated oligosaccharides” encompass fucosylated oligosaccharides with a certain homology of form since they contain a 2′-fucosyl-epitope, therefore a certain homology of function can be expected.

Within the context of the present invention, the expression “at least one fucosylated oligosaccharide” and “at least one N-acetylated oligosaccharide” means “at least one type of fucosylated oligosaccharide” and “at least one type of N-acetylated oligosaccharide”. In one embodiment the oligosaccharide of the present invention is a combination of 2-FL and LNnT in a ratio ranging from 1:20 to 2:1, preferably 1:15 to 1:1, most preferably of 1:10 to 1:2. In a particularly advantageous embodiment, this ratio is 2:1 or around 2:1.

In one embodiment the ratio of 2-FL:LNnT is 10:1.

Within the context of the present invention, the term “aqueous compositions” identifies liquid compositions which may be solutions and/or dispersions of the at least one oligosaccharide in an aqueous mean. In one embodiment the aqueous composition of the present invention can be obtained by a process as described in WO 2020/120426.

The term Full Enteral Feeding (FEF) is defined as end of parenteral nutrition and when minimum enteral intake of 150 ml/kg/day is attained. The term pre Full Enteral Feeding (pre-FEF) refers to the period from the day of birth till FEF is attained. Parenteral nutrition (consisting of fats, amino acids and sugars) is administered through intravenous route while enteral feeding (consisting for human milk or preterm formula) is administered through digestive tract. Study intervention was administered through the enteral route after enrolment (before FEF has been reached) until neonatal unit discharge.

Within the context of the present invention, the term “pH modulator” indicates a substance which is capable of affecting (i.e. decreasing, increasing or stabilizing) the pH of an aqueous solution. Non-limiting examples of pH modulators are strong and mild acids (organic or inorganic), acidic oligosaccharides, strong and mild bases (organic or inorganic) as well as buffers (organic or inorganic). Non limiting examples of organic acids are: Citric acid, Phosphoric acid, Lactic acid and sialic acid. Non limiting examples of inorganic bases are: potassium hydroxide (KOH) and sodium hydroxyde (NaOH). Non limiting examples of acidic oligosaccharides are sialic acid [N-acetyl-neuraminic acid (Neu5Ac)] or uronic acids (glucuronic acid, galacturonic acid). in one embodiment the pH modulator is a “buffer” and/or “buffering agent”. Non-limiting examples of buffering agents are: citric acid, acetic acid, phosphate salts (sodium or potassium). Non limiting examples of buffering solutions are: Phosphate buffer (based on 2 phosphates salts, for example sodium phosphate monobasic and sodium phosphate dibasic) and citrate-phosphate buffer (for example Mcllvaine buffer—based on citric acid and disodiumphosphate)

Within the context of the present invention, the term “fortifier” refers to a composition which comprises one or more nutrients having a nutritional benefit for infants.

By the term “milk fortifier”, it is meant any composition used to fortify or supplement either human breast milk, infant formula, growing-up milk or human breast milk fortified with other nutrients. Accordingly, the human milk fortifier of the present invention can be administered after dissolution in human breast milk, infant formula, growing-up milk or human breast milk fortified with other nutrients or otherwise it can be administered as a standalone composition.

When administered as a stand-alone composition, the human milk fortifier of the present invention can be also identified as being a “supplement”. In one embodiment, the milk fortifier of the present invention is a supplement.

By the term “human milk fortifier”, it is meant any composition used to fortify or supplement human breast milk, or human breast milk fortified with other nutrients. Within the context of the present invention, the expression “composition having a pH ranging from value X to value Y” identifies compositions having a pH range which has one specified value within the indicated range (extremes X and Y of the range being included) as well as compositions having a pH which varies within the indicated range (extremes X and Y of the range being included).

### Embodiments of the Invention

### Oligosaccharides

In one embodiment, the aqueous composition according to the present invention comprises two or more human milk oligosaccharides for use in use in promoting postnatal growth in preterm infants as early as 3 weeks after supplementation, wherein the composition is given as soon as possible after birth. The oligosaccharide(s) comprises 2′-FL, DFL, LNnT, LNT and combinations thereof.

In one embodiment, the present invention provides an aqueous composition as above defined which comprises 2′-FL and LNnT in a ratio of 10:1 for use in use in promoting postnatal growth in preterm infants as early as 3 weeks after supplementation, wherein the composition is given as soon as possible after birth.

In another embodiment, the present invention provides an aqueous composition at pH ranging from 4 to 7 as above defined which comprises 2′-FL and LNnT in a ratio of 10:1 wherein the composition is administered as soon as possible after birth between day 1 and 7 of life. In one embodiment the dosage amounts for pre-FEF ranges from 0.21-0.63 g/d and dosage amounts for FEF ranges from 0.43-0.82 g/d.

In one embodiment, the present invention provides an aqueous composition as above defined which comprises 2′-FL, DFL and LNT in a ratio of 10:1: 3.33.

In one embodiment, the aqueous composition according to the present invention comprises four human milk oligosaccharides is 2′-FL, DFL, LNnT and LNT.

In one embodiment of the present invention, the aqueous composition does not have sialylated oligosaccharides or probiotics.

### pH Range

In one embodiment, the aqueous composition according to the invention has a pH ranging from 4 to 7.

In a further embodiment, the aqueous composition according to the invention has a pH ranging from 5.8 to 6.3 or 5.9 to 6.2 or around 6.

### Aqueous Compositions

In one embodiment, the present invention provides an aqueous composition comprising at least one human milk oligosaccharide, wherein the pH of such aqueous composition ranges from 4 to 7, for example from 5.8 to 6.3 or from 5.9 to 6.2, and the composition doesn't comprise other nutrients in addition to the at least one oligosaccharide.

In one embodiment, the present invention provides an aqueous composition comprising at least one human milk oligosaccharides, a pH modulator and wherein the pH of such aqueous composition ranges from 5.5 to 6.5, for example from 5.8 to 6.3 or from 5.9 to 6.2, and the composition doesn't comprise other nutrients in addition to the oligosaccharide.

In one embodiment, the present invention provides an aqueous composition comprising at least one human milk oligosaccharides, a pH modulator, a buffering agent and wherein the pH of such aqueous composition ranges from 5.5 to 6.5, for example from 5.8 to 6.3 or from 5.9 to 6.2, and the composition doesn't comprise other nutrients in addition to the oligosaccharide.

In another embodiment, the present invention provides an aqueous composition comprising two human milk oligosaccharides, wherein the pH of such aqueous composition ranges from 5.5 to 6.5, for example from 5.8 to 6.3 or from 5.9 to 6.2, and the composition doesn't comprise other nutrients in addition to the oligosaccharides.

In another embodiment, the present invention provides an aqueous composition comprising two human milk oligosaccharides, a pH modulator and wherein the pH of such aqueous composition ranges from 5.5 to 6.5, for example from 5.8 to 6.3 or from 5.9 to 6.2, and the composition doesn't comprise other nutrients in addition to the oligosaccharides.

In another embodiment, the present invention provides an aqueous composition comprising two human milk oligosaccharides, a pH modulator, a buffering agent and wherein the pH of such aqueous composition ranges from 5.5 to 6.5, for example from 5.8 to 6.3 or from 5.9 to 6.2, and the composition doesn't comprise other nutrients in addition to the oligosaccharides.

In another embodiment, the present invention provides an aqueous composition comprising two human milk oligosaccharides, a pH modulator, a buffering agent and wherein the pH of such aqueous composition ranges from 5.5 to 6.5, for example from 5.8 to 6.3 or from 5.9 to 6.2, the oligosaccharides are 2′-FL and LNnT and the composition doesn't comprise other nutrients in addition to the oligosaccharides.

In one embodiment, the present invention provides an aqueous composition comprising at least one human milk oligosaccharide at a concentration ranging from 5 to 50% w/w of the composition.

In another embodiment, the present invention provides an aqueous composition comprising at least one human milk oligosaccharide at a concentration ranging from 8 to 35% w/w of the composition.

In a still further embodiment, the present invention provides an aqueous composition comprising at least one human milk oligosaccharide having a glucose unit at the reducing end at a concentration ranging from 10 to 30% w/w of the composition.

In one embodiment, the present invention provides an aqueous composition comprising at least one human milk oligosaccharides at a concentration ranging from 8 to 35% w/w of the composition, a pH modulator and wherein the pH of such aqueous composition ranges from 5.5 to 6.5, for example from 5.8 to 6.3 or from 5.9 to 6.2.

In one embodiment, the present invention provides an aqueous composition comprising at least one human milk oligosaccharides at a concentration ranging from 8 to 35% w/w of the composition, a pH modulator, a buffering agent and wherein the pH of such aqueous composition ranges from 5.5 to 6.5.

In another embodiment, the present invention provides an aqueous composition comprising two human milk oligosaccharides at a concentration ranging from 8 to 35% w/w of the composition, wherein the pH of such aqueous composition ranges from 5.5 to 6.5, for example from 5.8 to 6.3 or from 5.9 to 6.2.

In another embodiment, the present invention provides an aqueous composition comprising two human milk oligosaccharides at a concentration ranging from 8 to 35% w/w of the composition, a pH modulator, a buffering agent and wherein the pH of such aqueous composition ranges from 5.5 to 6.5, for example from 5.8 to 6.3 or from 5.9 to 6.2, the oligosaccharides are 2′-FL and LNnT.

In a further embodiment, the present invention provides an aqueous composition comprising five human milk oligosaccharides at a concentration ranging from 8 to 35% w/w of the composition, a pH modulator, a buffering agent and wherein the pH of such aqueous composition ranges from 5.5 to 6.5, for example from 5.8 to 6.3 or from 5.9 to 6.2, the oligosaccharides are 2′-FL, 3′-SL, 6′-SL, DFL and LNT.

In one embodiment, the present invention provides an aqueous composition comprising at least one oligosaccharide having a glucose unit at the reducing end at a concentration ranging from 8 to 35% w/w of the composition, wherein the pH of such aqueous composition ranges from 5.5 to 6.5, for example from 5.8 to 6.3 or from 5.9 to 6.2, and the composition doesn't comprise other nutrients in addition to the at least one oligosaccharide.

In another embodiment, the present invention provides an aqueous composition comprising two human milk oligosaccharides at a concentration ranging from 8 to 35% w/w of the composition, wherein the pH of such aqueous composition ranges from 5.5 to 6.5, for example from 5.8 to 6.3 or from 5.9 to 6.2, and the composition doesn't comprise other nutrients in addition to the oligosaccharides.

### Format

By having a liquid form, aqueous compositions according to the present invention present some particular benefits. For example, they may be more conveniently packed to deliver calibrated drops of a certain weight or volume.

In some embodiment, aqueous compositions of the present invention may be packed in single doses in such a way that calibrated drops of a certain weight or volume are delivered while avoiding contamination of the remaining liquid due to manipulation and subsequent uses.

In one embodiment, the liquid aqueous composition according to the present invention is presented in single dose units which are packed in plastic material. In one embodiment, such plastic material is flexible and squeezable. In one embodiment, such plastic material may be polypropylene (PP) or Polyethylene (PE). In one embodiment, polypropylene may be low density (LD PE) or high density (HD PE). In addition, aqueous compositions are easy to mix with compositions to be fortified, whereas the powder ones can, in some cases, form lumps.

### Supplements

In one embodiment, the aqueous composition according to the present invention and above described is a supplement. In such embodiment, the aqueous composition of the invention is administered as a standalone composition. In such embodiment, the aqueous composition of the invention is administered as a standalone composition and is packed in single doses.

### Fortifiers

In one embodiment, the aqueous composition according to the present invention is a milk fortifier. In such embodiment, the aqueous composition of the invention may be packed in single doses.

### EXPERIMENTAL SECTION

Objectives and Study: Low birthweight (LBW) preterm infants are susceptible to developmental programming of adverse health outcomes and abnormal ex-utero growth patterns. Poor early growth within the neonatal unit is associated with later developmental delays. This randomized, double-blind, placebo-controlled trial of LBW preterm infants evaluated effects of HMO supplementation on feeding tolerance, growth and safety from 7 centers in France.

Methods: Preterm infants between 27 and 33 weeks of gestational age with birth weight<1700 g, who are younger than 7 days of age were randomized as early as possible after birth to receive HMO supplement comprising of 2′FL and LNnT (n=43) in 10:1 ratio (0.34 and 0.034 g/kg body weight/day, respectively) or an isocaloric Placebo supplement (n=43) consisting of only glucose (0.14 g/kg/day) from enrolment until discharge from the neonatal unit. Anthropometric z-scores during neonatal unit stay (weight, length and head circumference) were calculated using the Fenton standards.

Results: Mean chronological age at enrolment in HMO and Placebo groups were 6.3 and 6.2 days, respectively. There was no difference in weight z-scores between HMO and Placebo groups at any time point (overall treatment difference 0.03, 95% Cl −0.13, 0.20, p=0.682). Length z-scores were significantly higher in HMO group vs. Placebo at Day(D) 14 (treatment difference 0.29, 95% CI 0.02, 0.56, p=0.037) and D21(treatment difference 0.31, 95% Cl 0.02, 0.61, p=0.037). Head circumference z-score was significantly higher in HMO group vs. Placebo at Discharge (treatment difference 0.42, 95% Cl 0.12, 0.71, p=0.007). There were no adverse effects of HMO supplementation; serious necrotizing colitis occurred in 4.5% and 2.4% of subjects in HMO and Placebo groups, respectively, and only one case in the Placebo group was assessed to be related to study intervention. Among 50% and 42.9% of subjects who experienced Infections and Infestations adverse events in HMO and Placebo groups, respectively, none of them were deemed to be serious.

Conclusion: HMO supplementation is safe and well-tolerated in preterm infants. When given as soon as possible after birth, HMO supplementation supported age-appropriate z-scores in length and head circumference. As a result, HMO supplement may support improved early postnatal growth, especially in head growth, which is well-established determinant of longitudinal cognitive development of preterm infants.

1. A method for use in promoting postnatal growth in preterm infants as early as 3 weeks after supplementation comprising administering an aqueous composition comprising at least one human milk oligosaccharide to a preterm infant as soon as possible after birth.

2. Method according to claim 1 wherein the postnatal growth is increase in head circumference and/or length.

3. Method according to claim 1 wherein the postnatal growth is increase in length observed at onset of 14 days after achieving full enteral feeding (FEF) of the said composition as compared to the preterm infants receiving placebo only for the same period.

4. Method according to claim 1 wherein the postnatal growth is increase in length observed between 14 to 21 days after achieving FEF of the said composition as compared to the preterm infants receiving placebo only for the same period.

5. Method according to claim 1 wherein the postnatal growth is gain in head circumference observed at neonatal unit discharge (approx. 6 weeks after birth) as compared to the preterm infants receiving placebo only for the same period.

6. Method according to claim 1 wherein the pH of said composition ranges from 4 to 7.

7. Method according to claim 1 wherein the composition is a milk fortifier or a supplement.

8. Method according to claim 1 wherein the composition is administered as soon as possible after birth between day 1 to day 7 of life.

9. Method according to claim 1 wherein the composition further comprises a buffering agent.

10. Method according to claim 1 wherein the at least one oligosaccharide is selected from the group consisting of: 2′-FL, DFL, LNnT and LNT.

11. Method according to claim 1 wherein the composition comprises only two oligosaccharides as human milk oligosaccharides comprising 2′-FL and LNnT at ratio 10:1.

12. Method according to claim 1 wherein the composition does not comprise other nutrients in addition to the at least one oligosaccharide.

13. Method according to claim 1 wherein the composition comprises oligosaccharides at a concentration ranging from 8 to 35% w/w of the composition.

14. Method according to claim 1 for use in supporting brain-catchup growth in preterm infants.
